(19) United States
(12) Patent Application Publication
(10) Pub. No.: US 2014/0301722 A1
(43) Pub. Date: Oct. 9, 2014

(54) Devices and Methods for Emanating Liquids

(71) Applicants: Helen Stephenson, Hull (GB); Richard Koontz, Hull (GB); Paul Newton, Hull (GB)

(72) Inventors: Helen Stephenson, Hull (GB); Richard Koontz, Hull (GB); Paul Newton, Hull (GB)

(73) Assignee: Reckitt & Colman (Overseas) Limited, Slough, Berkshire (GB)

(21) Appl. No.: 14/128,036

(22) PCT Filed: Jun. 25, 2012
(86) PCT No.: PCT/GB2012/051480

§ 371 Date: Feb. 28, 2014

(30) Foreign Application Priority Data
Jun. 24, 2011 (GB) 1110698.6

(51) Int. Cl. A61L 9/12; A61L 9/03

(52) U.S. Cl. CPC A61L 9/12; A61L 9/037
USPC 392/395; 239/44

(57) ABSTRACT

A liquid dispensing system is described comprising a universal refill of liquid and an emanation device; wherein the refill comprises: a housing having an inner volume and an outer surface; at least one reservoir in the inner volume of the housing for holding the liquid; at least one aperture in the housing sealed by a sealing means; and at least one wick provided on or connected to the outer surface of the housing, wherein said wick(s) is not extend to the inner volume of the housing; and wherein the emanation device is selected from: an emanation device configured to spray the liquid; an emanation device configured to evaporate the liquid by electrically powered means; an emanation device configured to evaporate the liquid by passive, non-electrically powered means; an emanation device configured to emanate the liquid by two or more of spraying the liquid, evaporating the liquid by electrically powered means and/or evaporating the liquid by passive, non-electrically powered means;

characterised in that each emanation device is provided with a chassis configured to receive and releasably retain the universal refill and the universal refill is operationally compatible with each emanation device.

### FIELD OF THE INVENTION

The present invention relates to devices and methods for improved airborne delivery of liquids containing one or more active materials wherein the active material comprises at least one of: a fragrance; an insecticide; a fungicide; a pesticide; a sanitising material; and/or a pharmaceutical.

### BACKGROUND

Liquids, and commonly volatile liquids, containing one or more active materials wherein the active material comprises at least one of: a fragrance; an insecticide; a fungicide; a pesticide; a sanitising material; and/or a pharmaceutical are delivered within the domestic environment via a variety of mechanisms. Devices are available with heaters disposed therein to increase the rate of emanation from a surface saturated with the liquid, such a surface could be a wick saturated with a fragranced liquid and the heater is located adjacent the wick surface and nearby a chimney to heat the liquid on the wick surface and cause it to more readily evaporate and disseminate into the surrounding environment through the chimney.

Alternatively such liquids can be loaded into an aerosol canister wherein the canister holds the liquid under pressure and when a valve is opened the liquid is forced out. The liquid is provided with a propellant which evaporates inside the canister to maintain an even pressure and, outside the canister, assist with the mechanical break up of the liquid by evaporating rapidly. Suitable propellants include volatile hydrocarbons such as propane, butane or isobutane.

Aerosols generally provide a satisfactory spray performance but since they require manual operation by a user, they are not considered to be particularly convenient for routine use.

Automatic aerosol activation devices exist for operation with metered dose aerosols containing a single phase liquid and propellant mixture. These devices are operable to periodically actuate the aerosol to cause a dose of the liquid to be sprayed.

Whilst the automation of the aerosol spraying devices overcomes the problem of manual operation of an aerosol canister, the use of aerosols containing such propellants is becoming increasingly less desirable since these volatile hydrocarbons carry the disadvantage of being flammable, the rising cost of oil is rendering them increasingly expensive and they also carry the further disadvantage of being an atmospheric source of carbon which is deemed to not be environmentally sensitive.

There are other known emanation devices using alternative emanation methodologies such as nebulisation techniques using a piezo-actuated apparatus for instance.

All of the known emanation devices and methodologies of emanation possess advantages and disadvantages with respect to each other that will be appreciated by a person skilled in the art as well as the ultimate end user. However, invariably the consumer is faced with a dilemma when purchasing any particular emanation device as the emanation method employed by the emanation device under consideration is not the only factor the consumer must also consider, the consumer must also consider the availability and range of refills of liquids to be used with said device. In the field of air freshening in particular where the range of fragrances the consumer can select for use with the device is a key point of purchase consideration for the consumer, a small range of fragrances and/or fear that the refill range may not be supported in the long term may affect the consumer's true freedom of choice. The present invention addresses this and other problems.

### SUMMARY OF INVENTION

According a first aspect of the present invention there is provided therefore a universal refill of liquid containing one or more active materials wherein the refill comprises:

a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) is not extend to the inner volume of the housing; and
wherein the refill is configured to allow the liquid to be extracted by at least two different methods of liquid extraction.

According a second aspect of the present invention there is provided therefore a universal refill containing a liquid for use with at least two different types of emanation device;

wherein the universal refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) is not extend to the inner volume of the housing; and
wherein each type of device emanates the liquid from the universal refill via a different mechanism to each other type of emanation device and wherein said at least two different types of emanation device each comprise a chassis configured to receive and releasably retain the universal refill.

According to a third aspect of the present invention there is provided a liquid dispensing system comprising a universal refill of liquid and an emanation device; wherein the refill comprises:

a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) is not extend to the inner volume of the housing; and
wherein the emanation device is selected from: an emanation device configured to spray the liquid; an emanation device configured to evaporate the liquid by electrically powered means; an emanation device configured to evaporate the liquid by passive, non-electrically powered means; an emanation device configured to emanate the liquid by two or more of spraying the liquid, evaporating the liquid by electrically powered means and/or evaporating the liquid by passive, non-electrically powered means;
characterised in that each emanation device is provided with a chassis configured to receive and releasably retain the universal refill and the universal refill is operationally compatible with each emanation device.

According to a fourth aspect of the present invention there is provided a liquid dispensing system comprising a universal refill of liquid and an emanation device; wherein the refill comprises:

a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) is not extend to the inner volume of the housing; and
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill and the universal refill is operationally compatible with each emanation device.

Within the context of the present invention the term “operationally compatible” is to be understood to require that the refill can be received within the chassis of an emanation device and permit the device to extract the liquid from the refill and emanate same into the surrounding environment.

According to a fifth aspect of the present invention there is provided a liquid dispensing device for use with a universal refill of liquid and an emanation device; wherein the refill comprises:

a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) is not extend to the inner volume of the housing;
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill.

Preferably the universal refill is provided with securing means which are configured, in use, to cooperate with the chassis and/or the emanation device to permit secure engagement therewith. Preferably the securing means form a part of the external surface of the housing.

Unlike all prior art refills which contain a wick in contact either completely or partially with the liquid, in the present invention the wick(s) does not extend to the inner volume of the housing and, thus, cannot be in direct liquid communication with any liquid contained within the reservoir. The at least one wick of the refill of the present invention is not physically able to contact any liquid in the reservoir and can only be used to as a platform to emanate any liquid therefrom once any liquid from the reservoir is transported via a separate transport mechanism from within the inner volume of the housing to the outer surface of the housing where the wick(s) is located. The arrangement of the present invention carries numerous advantages as a consequence. A principal advantage is that no fractionation of the liquid occurs since the wick is being used for emanation purposes only rather than for transport and emanation of the liquid, such as transporting the liquid from the reservoir within the housing and out of the housing to the extremity of the wick which typically protrudes above the exterior of the housing before emanating same to the surrounding environment. The drawback of this transport and emanation is the phenomenon of fractionation, or a variant thereof, which can result when the liquid to be emanated comprises numerous components having different molecular weights and volatilities, the resultant effect can be that the light MW components and/or more volatile components are transported quicker to the end of the wick where emanation typically occurs leaving the heavier MW and/or lower volatility components to be emanated latterly. Such fractionation is particularly noticeable when dealing with a fragranced liquid as the ‘lighter/heady’ notes of the fragrance emanate off quickly leaving the ‘heavier/base’ notes to emanate off later resulting in an experience which is non-uniform and not a true representation of the fragrance the liquid manufacturer intended the user to experience during use. Although potentially less noticeable from a consumer perspective, a further drawback could be when attempting to emanate an insecticide or the like containing one or more active insecticide materials, wherein any fractionation or the like as discussed above can result in non-uniform delivery of the active materials and, potentially, non-uniform levels of protective insecticide in the atmosphere surrounding the refill.

Although a plurality of apertures may be provided, in one preferred arrangement the universal refill housing has a single aperture which is, preferably, located in an upper wall of the housing. It is to be understood that reference to an “upper wall” is made relative to the other walls of the universal refill housing purely for the purpose of spatially describing the refill and, unless otherwise stated, is not to be understood as imparting any restrictive orientation on the refill itself.

The sealing means may be provided by any suitable closure mechanism which permits any liquid contained within the reservoir to be safely and conveniently transported until it is needed. Preferably however, the sealing means is provided by at least one valve. Where a valve(s) is present, said valve(s) is preferably configured to be automatically resealable when not being held open.

The at least one valve may be provided by an automatically resealable valve. The valve may be provided in the form of a self-sealing liquid-tight valve, such as a silicone valve, septum valve or the like. Alternatively the valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In one preferred arrangement however at least two valves are provided in the form of a downstream valve (i.e. the valve closest to the reservoir) and an upstream valve. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 1-2000 μm, and more preferably with a diameter of between 50-1500 μm, and even more preferably with a diameter of between 100-1000 μm. The relatively narrow diameters of the preferred ranges permits the annular ring to form a sealing engagement with a capillary tube or the like that enters the ring. Alternatively, the opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Said ranges of relatively larger diameters permits the annular ring to form a sealing engagement with an extraction limb that enters the ring; said extraction limb may house one or more liquid conduits and/or one or more capillary tubes or the like. Whilst the annular ring is preferably provided in a circular shape other shapes may be permissible providing the shape is capable of performing the sealing function required of it, such shapes include substantially circular shapes, oval shapes, diamond shapes and such like.

The downstream valve is preferably operable to prevent any fluid within the refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In one preferred embodiment the downstream valve and the upstream valve are formed as separate pieces that are held adjacent but spaced apart from each other. In a most preferred embodiment however the downstream valve and the upstream valve are formed as a one piece component.

In an alternatively preferred embodiment, the at least one valve is provided in the form of a duckbill valve, wherein the extends into the inner volume of the housing to be operable to be opened when a body, such as a capillary tube or the like, is inserted whilst otherwise be held closed to prevent the loss of any liquid contained therein during transport or storage of the refill. The universal refill may be provided with one or more vent holes in the housing, said vent holes being provided in the form of a one-way valve that is in communication with the reservoir but does not facilitate a liquid pathway therefrom rather it solely permits air to enter the reservoir from the outside of the refill to prevent or reduce any build up of negative air pressure within the refill as liquid is removed therefrom. The vent hole(s) may be covered with a gas permeable membrane. Preferably however, the vent hole(s) is not covered and is sized such that the liquid may not escape therefrom or that the rate of escape would be sufficiently low as to not be relevant for the safe operation of the refill with a device configured to operate with the refill to emanate liquid obtained therefrom.

The universal refill may further comprise one or more diptubes therein. Said diptube(s) may be in communication with the vent hole and/or in communication with the aperture.

Said at least one valve could be adjustable to affect the flow rate of the liquid therethrough. The adjustability may be facilitated manually by a user and/or due to an automated function of a device to which the refill is connected and said automated function may be controlled automatically by the device or may be in response to a user input into the device. Adjustability may be particularly useful when the device is configured to emanate the liquid passively and/or extract the liquid passively such as by gravity feed.

In an alternatively preferred arrangement at least two apertures may be provided in the universal refill housing, and even more preferably two apertures are provided. The apertures are preferably located in an upper wall of the refill housing. It is to be understood that reference to an “upper wall” is made relative to the other walls of the housing purely for the purpose of spatially describing the refill and, unless otherwise stated, is not to be understood as imparting any restrictive orientation on the refill itself.

Each aperture is preferably sealed by the at least one valve wherein each valve is preferably a single automatically resealable valve. Said single valve may be provided in the form of a self-sealing liquid-tight valve, such as a silicone valve, a septum valve or the like. Alternatively said single valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In a preferred arrangement however at least one the apertures is sealed by at least two valves provided in the form of a downstream valve (i.e. the valve closest to the reservoir) and an upstream valve. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 1-2000 μm, and more preferably with a diameter of between 50-1500 μm, and even more preferably with a diameter of between 100-1000 μm. The relatively narrow diameters of the preferred ranges permits the annular ring to form a sealing engagement with a capillary tube or the like that enters the ring. Alternatively, the opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Said ranges of relatively larger diameters permits the annular ring to form a sealing engagement with an extraction limb that enters the ring; said extraction limb may house one or more liquid conduits and/or one or more capillary tubes or the like. Whilst the annular ring is preferably provided in a circular shape other shapes may be permissible providing the shape is capable of performing the sealing function required of it, such shapes include substantially circular shapes, oval shapes, diamond shapes and such like.

The downstream valve is preferably operable to prevent any fluid within the universal refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In a preferred arrangement however all of the apertures are sealed by at least two valves provided in the form of a downstream valve (i.e. the valve closest to the reservoir) and an upstream valve.

In one preferred embodiment the downstream valve and the upstream valve are formed as separate pieces that are held adjacent but spaced apart from each other. In a most preferred embodiment however the downstream valve and the upstream valve are formed as a one piece component.

The wick(s) is preferably provided on or connected to the outer surface of the upper wall of the housing adjacent the aperture(s). This arrangement is advantageous as it minimises the distance the liquid has to travel from the reservoir to the wick before being emanated, thus improving the response time of the wick when transitioning between emanating and non-emanating functionality.

Preferably however, the wick(s) is located on or connected to the outer surface of the housing such that when the refill is located in a position to extract and emanate liquid contained within the reservoir, then at least one wick is located substantially level with or, most preferably, above the liquid level in the reservoir. Such an arrangement is particularly preferable when extracting liquid from the reservoir via capillary action as an optimum transfer rate of liquid from the reservoir to the wick(s) can be achieved providing the relative height of wick to the height of the liquid level in the reservoir is controlled such that the wick(s) is either substantially level with or slighter higher than the liquid level.

In one embodiment one or more wicks may be provided on or in contact with numerous outer surfaces of the housing such that regardless of the position of the refill when extraction and emanation is to occur at least one wick will be located substantially level with or higher than the liquid level in the reservoir.

Although a plurality of wicks may be provided, preferably a single wick is provided.

The wick may be provided on a collar which is connected to the outer surface of the housing and the collar may be sized to extend away from the outer surface of the housing. However, the wick is preferably sized to remain within contact with the upper wall of the housing without hanging over the edge thereof. Alternatively, the wick may contact both the outer surface of the upper wall of the housing and extend over the edge of the upper wall to also contact one or more side walls of the housing.

The wick(s) may be made of any material that is capable of wicking and emanating a liquid, such suitable materials include plastics materials such as cintered polyethylene, cellulose, woods such as balsa or bamboo, reeds such as rattan.

The reservoir(s) may be provided in the form of a flexible bag which is capable of collapsing inwardly as liquid is extracted therefrom.

The chassis may be formed as a separate component to the emanation device and may be mounted on or in the device to receive and releasably retain the universal refill. Alternatively or additionally, the chassis may be formed in an exterior surface of the emanation device (such as a device housing). Alternatively or additionally the chassis may be formed within the interior of the device and a suitably shaped aperture is provided to permit access to the interior of the housing by the universal refill. Alternatively or additionally the chassis may be formed within the interior of the device and the device is provided with a door which may be opened to permit access to the interior of the housing by the universal refill.

Where the emanation device is to pump the liquid from the reservoir(s) of the universal refill the emanation device may be provided with one or more liquid conduits that are connected at one end to a pumping means housed within the device and open at the other end to contact the liquid. The liquid conduit(s) are preferably sized to extend through the, or each, valve system in the universal refill and into the reservoir(s) therewithin when the refill is engaged with the chassis. In use, the pump may be operable via conventional pumping mechanisms to create negative pressure within the liquid conduit(s) in order to draw the liquid into the conduit(s) and transport same toward the pump. Once the liquid is in the device it may be emanated therefrom in any of a number of convention mechanisms, for instance the pumped liquid may be transported to an open emanation plate in combination with a heater to promote evaporation of the liquid, or the liquid may be transported to a nebuliser and it is atomised thereby, etc.

Where the emanation device is to blow the liquid from the reservoir(s) of the universal refill the emanation device may be provided with one or more blown air conduits that are connected at one end to a blowing means housed within the device and open at the other end to permit blown air to exit therefrom and with one or more liquid conduits that are open at one end to contact the liquid and connected to the device at the other end. The blown air conduit(s) are preferably sized to enter through one valve system of the universal refill and the liquid conduit(s) are preferably sized to extend through one or more of alternate valve systems in the refill (i.e. —the blown air conduit(s) and the liquid conduit(s) do not use the same valve system) and wherein both types of conduits extend into the reservoir(s) within the refill when said refill is engaged with the chassis. In use, the blowing means may be operable to blow air along the blown air conduit and into the reservoir(s) in order to pressurise the head space (i.e. —the head space is the space within the reservoir(s) not taken up by the liquid) and, when required by the device, force liquid into the liquid conduit(s) and toward the device. liquid transported along the liquid conduit(s) may be emanated therefrom in any of a number of convention mechanisms, for instance the liquid may be sprayed directly from the device under the force of said transportation and/or in combination with additional blown air from the blowing means, the liquid may be transported to a nebuliser and for it to be atomised thereby, etc.

Alternatively, where the universal refill is provided with a single aperture and single valve system, the blown air conduit and liquid conduit could be integrated such that the blown air conduit extends along a central core of the liquid conduit with the blown air conduit being provided with the greater length such that it can extend further into the refill and such that the conduits can enter the single aperture and valve system; the blown air conduit could be adjacent the liquid conduit however. In this arrangement the blown air conduit may act as described above by blowing air into the headspace of the refill to force liquid into the liquid conduit and into the device for emanation therefrom. As a further alternative, an integrated conduit can be provided with one conduit extending along a central core of the other conduit such that the conduits can both enter a single aperture and valve system, wherein the one conduit is a liquid conduit which has air blown across the top thereof to draw the liquid along the conduit via the Venturi effect and/or capillary effect, whilst the other conduit allows air to be returned to the reservoir to prevent choking of the liquid flow.

Where the emanation device is to wick the liquid from the reservoir(s) of the universal refill the emanation device may be provided with one or more wicks that are connected at one end to the emanation device and configured at the other end to contact the liquid and uptake same. The wick(s) are preferably sized to extend through the valve system(s) in the universal refill and into the reservoir(s) therewithin when the refill is engaged with the chassis. In use, the wick(s) may be operable to passively uptake liquid from the reservoir(s) and transport same towards the other end of the wick(s). Once the liquid is within the wick(s) may be emanated from the device in any of a number of convention mechanisms, for instance the liquid may be emanated directly from the wick(s) passively and/or in combination with powered means to assist in the evaporation such as an electrically powered heater, or the liquid may be transported to along the wick(s) to a nebuliser and it is atomised thereby, etc.

A preferred wicking arrangement is provided however where the emanation device is to transport the liquid from the reservoir(s) of the universal refill to the emanation device via capillary action the device may be provided with one or more capillary tubes that contact the liquid and uptake same at one end and terminate at their other end adjacent to or in contact with the wick on the exterior surface of the universal refill housing. The capillary tube(s) are preferably sized to extend through the valve system(s) in the universal refill and into the reservoir(s) therewithin when the refill is engaged with the chassis. In use, the capillary tube (s) may be operable to passively uptake liquid from the reservoir(s) and transport same towards the other end of the tube(s). If the liquid transported along the capillary tube(s) is transferred to the wick on the exterior of the refill housing a heater can be directed toward to the wick to accelerate the emanation of the liquid therefrom. Alternatively the liquid transported via the capillary tube(s) may be transported to the emanation device may then be emanated therefrom in any of a number of convention mechanisms, for instance the liquid may be transported to an open emanation plate in combination with a nebuliser to be atomised thereby, etc.

Where the emanation device is to extract the liquid from the reservoir(s) of the universal refill toward the emanation device by a gravity feed mechanism the emanation device may be provided with one or more liquid conduits that are connected at one end to the device and open at the other end to contact the liquid. The liquid conduit(s) are preferably sized to extend through the valve system(s) in the universal refill and into the reservoir(s) therewithin when the refill is engaged with the chassis. The chassis is orientated such when the universal refill is engaged therewith the valve system(s) of the refill are located closer to a supporting surface on which the device is held such that once the valve(s) are opened by the liquid conduit(s) the force of gravity will cause liquid to travel from the refill toward the device. In use, the liquid conduit(s) may be operable to simply open the valve system(s) to permit the liquid to flow from the reservoir toward the device to emanate the liquid by any of a number of convention emanation mechanisms, for instance the liquid flow into an open emanation plate in combination with a heater to promote evaporation of the liquid, or the liquid may flow toward a nebuliser for it to be atomised thereby, etc.

The emanation device and/or chassis may be provided with securing means which are configured to engage with the universal refill to permit a secure engagement of the universal refill to the chassis. Preferably the releasable engagement between the universal refill and the chassis is provided with a positive feedback for the user; such as a “push-click” mechanism wherein the user pushes the refill against the chassis until an audible “click” sound is achieved and the refill is secured relative to the chassis, and to disengage the universal refill from the chassis the user pushes the refill against the chassis until a further “click” sound is achieved accompanied by a physical sensation of the refill being pushed away from the chassis and the user may separate the refill from the device.

The universal refill and emanation devices are preferably provided with a device-to-refill recognition system whereby the device is able to recognise the level of liquid remaining in the refill and/or the type of liquid in the refill and/or whether the refill is from a known source. The recognition system may be provided by the device possessing a physical characteristic in the refill housing which interacts with a portion of the chassis and/or device. The recognition system may comprise a moulded feature in the reservoir(s) which may have a communication component embedded therein which can communicate with a corresponding communication component within the device and/or the moulded feature may act as a convenient means to for the device to make reference against when determining the level of liquid in the reservoir(s). The recognition system may be provided with an identification means being applied to and/or in the refill housing such as a visual code, an RFID tag, printed electrical components, magnets or the like. The recognition system may be provided by any combination of the aforesaid recognition systems.

Any of the features described herein may be combined with any of the above aspects in any combination.

### BRIEF DESCRIPTION OF THE DRAWINGS

Embodiments of the invention will now be described, by way of example only, with reference to the following drawings in which:

FIG. 1 shows a perspective view of one embodiment of the refill;

FIG. 2 shows a diagrammatic sectional view of the refill with a capillary tube of a first emanation device engaged therewith;

FIG. 3 shows a diagrammatic sectional view of the refill with a capillary tube of a second emanation device engaged therewith;

FIG. 4 shows a diagrammatic sectional view of the refill with a capillary tube of a third emanation device engaged therewith;

FIG. 5 shows a perspective sectional view of the refill with an extraction limb of a first emanation device engaged therewith;

FIG. 6 shows a perspective sectional view of the refill with an extraction limb of a second emanation device engaged therewith;

FIG. 7 shows a perspective sectional view of the refill with an extraction limb of a third emanation device engaged therewith; and

FIG. 8 shows a sectional view of the refill valve.

### DESCRIPTION OF AN EMBODIMENT

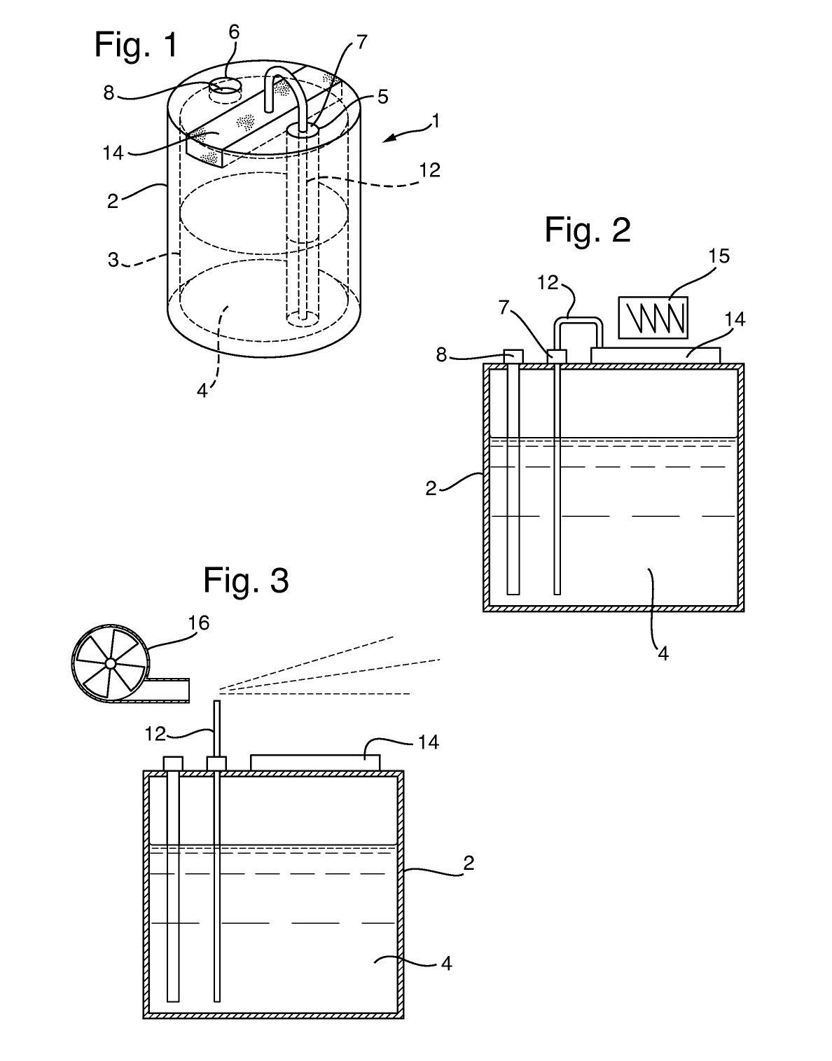

FIG. 1 shows a preferred embodiment of a refill 1 according to the present invention. The refill 1 comprises a housing 2 that surrounds and contains a reservoir 3 of liquid 4 therein. Access to the interior of the refill 1 and the reservoir 3 is provided via one of two apertures 5, 6 which is each sealed by a valve 7,8 (not shown in detail). A capillary tube 12 is sealingly engaged with valve 7.

In a preferred arrangement the tube 12 is part of an emanation device that is introduced into the valve 7 and extends toward the base 13 of the reservoir 3. The end of the tube remote from the base 13 is bent through 180° into a U-shape such that it terminates above a wick 14 which is provided entirely on an outer surface of the refill housing 2. The capillary tube 12 is hollow and has an internal diameter of 100 μm+/−50 μm. This diameter permits the liquid 4 to be transported at a desired rate from the reservoir 3 to the wick 14.

FIG. 8 shows the valve 7,8 in greater detail. Each valve 7,8 is an automatically resealable valve system comprising an open annular ring valve 9 suspended by a flexible annular skirt 10 which forms the upstream valve and the valve system further comprises a downstream valve in the form of a rubberised slit valve 11. The downstream slit valve 11 provides a fluid tight closure to prevent the liquid in the reservoir from escaping.

FIG. 2 shows how an emanation device can interact with one or more of the valves 7,8 of the refill 1. The hollow capillary tube 12 is provided operatively connected to the emanation device (the full device is not pictured). The tube 12 is generally elongate and provided at an upper end with a bent U-shape. Preferably the tube is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In the arrangement shown in FIG. 2 the tube 12 terminates at the end of the U-shape by contacting the wick 14. The wicking action of the wick 14 in combination with the capillary action within the tube 12 will drive the transport of the liquid 4 to the wick 14. Alternatively, the tube 12 could terminate adjacent the wick 14, and preferably just above the wick such that a drop will form at the end of the tube 12 and will grow in size until it reaches a critical mass where it can no longer adhere to the tube and falls on to the wick 14. Although not shown in FIG. 2, the device may also open valve 8 in order to permit air to be returned to the reservoir 3 and thus prevent any retarding of the transport of liquid out of the refill 1 due to build up of negative pressure as liquid 4 leaves the reservoir 3.

A heater 15 in the device is located adjacent the wick 14 to, in use, direct heat toward the wick and accelerate the volatilisation of the liquid therefrom and out of an exit port (not shown) in the device and into the surrounding environment.

FIG. 3 shows an emanation device (the full device is not pictured) that uses an air pump 16 to aid the extraction of the liquid 4 from the reservoir 3. A hollow capillary tube 12 is provided operatively connected to the emanation device. The tube 12 is generally elongate with an upper end that terminates adjacent the air pump 16. Preferably the tube is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In addition to the transporting of liquid via capillary action, the air pump 16 is configured to blow air across the top of the tube 12 to cause liquid to travel up the conduit by the Venturi effect as well. The flow of air is additionally instrumental in forcing the liquid against a mechanical break up means (not shown) and out of an exit port in the device into the surrounding environment. Although not shown in FIG. 3, the device may also open valve 8 in order to permit air to be returned to the reservoir 3 and thus prevent any retarding of the transport of liquid out of the refill 1 due to build up of negative pressure as liquid 4 leaves the reservoir 3.

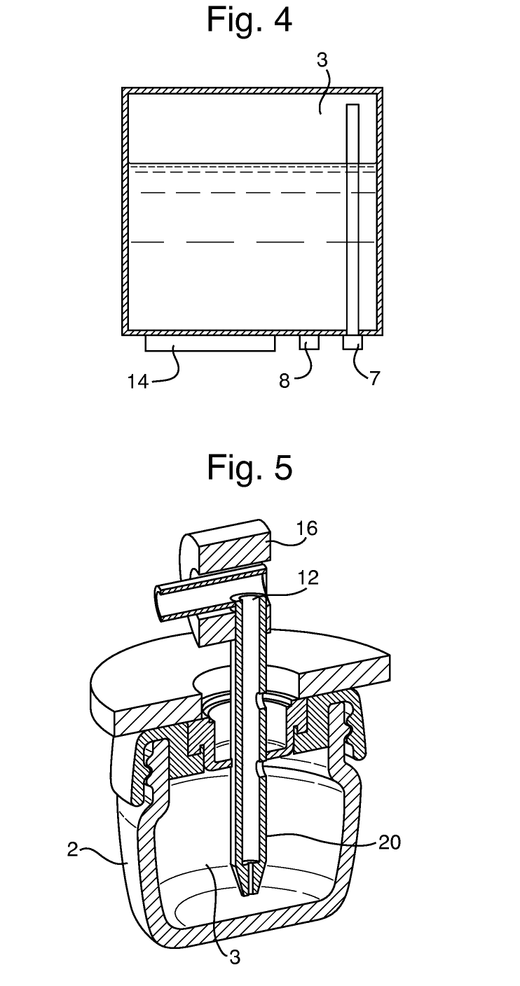

A gravity-feed emanation device (full device not pictured) is shown in FIG. 4. In this arrangement the refill 1 is loaded into the device in an inverted orientation. The tube 12 is generally elongate with an upper end that terminates adjacent toward an inner surface of the reservoir remote from the valves 7,8. Preferably the tube 12 is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. To extract the liquid 4 from the refill the device has an additional extraction means (not shown) that opens valve 8 to permit the liquid to flow in a downward direction powered by gravity and into the device. The tube 12 also air to flow into the reservoir in the head-space formed by the dropping liquid level to prevent any retarding of the transport of liquid out of the refill 1 due to build up of negative pressure as liquid 4 leaves the reservoir 3.

Whereas FIGS. 2-4 show a refill 1 having two valves 7,8, FIGS. 5-7 shown a refill with a single valve 7 and how emanation devices using differing extraction and emanation methods can interact with same.

In FIG. 5 an emanation device (the full device is not pictured) that uses an air pump 16 to aid the extraction of the liquid 4 from the reservoir 3 is shown. The device is provided with a hollow and elongate extraction limb 20 that surrounds a hollow capillary tube 12. The tube 12 is also elongate with an upper end that terminates adjacent the air pump 16. Preferably the limb 20 is sized to have a diameter that is slightly wider than the diameter of the ring valve 9 such that, in use, when the end of the limb 20 is pushed through the ring valve 9 a liquid-tight connection is made between the limb 20 and the ring valve 9. On travelling further into the valve 7 the end of the limb 20 will meet the slit valve 11 and force it open. Since the limb 20 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In addition to the transporting of liquid via capillary action, the air pump 16 is configured to blow air across the top of the tube 12 to cause liquid to travel up the conduit by the Venturi effect as well. The flow of air is additionally instrumental in forcing the liquid against a mechanical break up means (not shown) and out of an exit port in the device into the surrounding environment. Although not shown in FIG. 5, the refill 1 may be provided with a vent hole to permit air to return to the refill to prevent any retarding of the transport of liquid out of the refill 1 due to build up of negative pressure as liquid 4 leaves the reservoir 3.

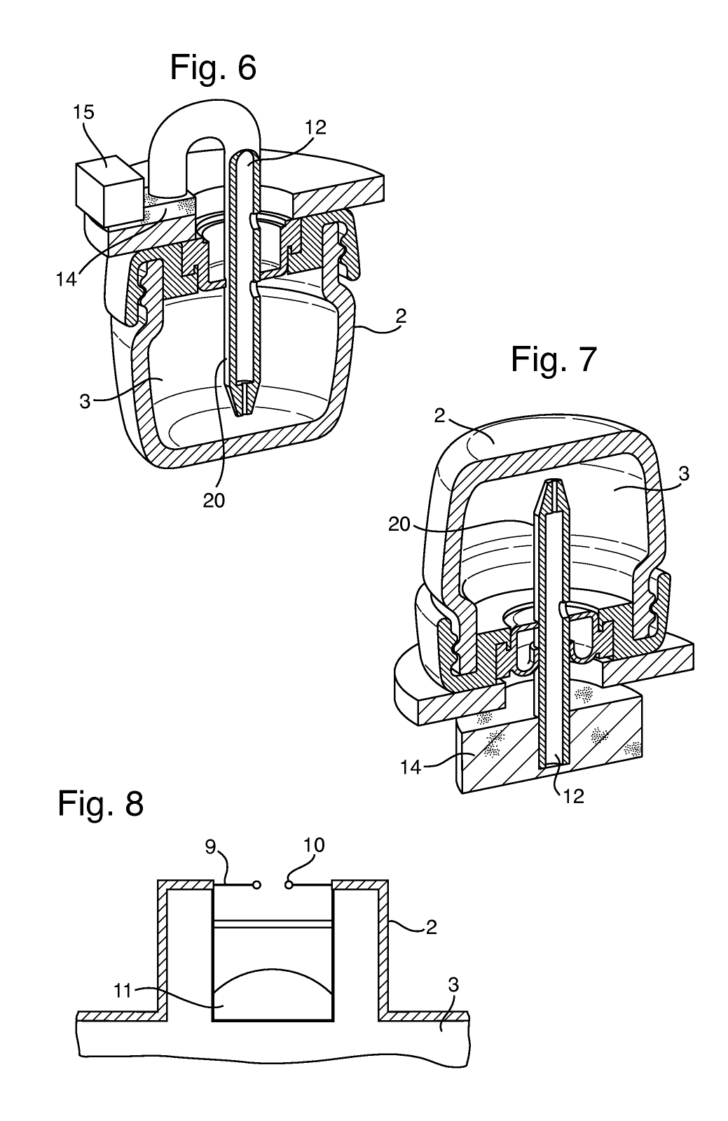

FIG. 6 shows a device that uses a combination of capillary action and a heater to extract and emanate liquid from the refill. The device (the full device is not pictured) is provided with a hollow extraction limb 20 that surrounds the hollow capillary tube 12. The extraction limb 20 is generally elongate and the tube 12 is also generally elongate but provided at an upper end with a bent U-shape. Preferably the limb 20 is sized to have a diameter that is slightly wider than the diameter of the ring valve 9 such that, in use, when the end of the limb 20 is pushed through the ring valve 9 a liquid-tight connection is made between the limb 20 and the ring valve 9. On travelling further into the valve 7 the end of the limb 20 will meet the slit valve 11 and force it open. Since the limb 20 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In the arrangement shown in FIG. 6 the tube 12 terminates at the end of the U-shape by contacting the wick 14. The wicking action of the wick 14 in combination with the capillary action within the tube 12 will drive the transport of the liquid 4 to the wick 14. Alternatively, the tube 12 could terminate adjacent the wick 14, and preferably just above the wick such that a drop will form at the end of the tube 12 and will grow in size until it reaches a critical mass where it can no longer adhere to the tube and falls on to the wick 14.

A heater 15 in the device is located adjacent the wick 14 to, in use, direct heat toward the wick and accelerate the volatilisation of the liquid therefrom and out of an exit port (not shown) in the device and into the surrounding environment.

Although not shown in FIG. 6, the refill 1 may be provided with a vent hole to permit air to return to the refill to prevent any retarding of the transport of liquid out of the refill 1 due to build up of negative pressure as liquid 4 leaves the reservoir 3.

A gravity-feed emanation device (full device not pictured) is shown in FIG. 7. In this arrangement the refill 1 is loaded into the device in an inverted orientation. The device is provided with a hollow and elongate extraction limb 20 containing at least one perforation (not shown) therein to permit access to the hollow interior of the limb 20. The extraction limb 20 is provided with a hollow capillary tube 12 therein. Preferably the limb 20 is sized to have a diameter that is slightly wider than the diameter of the ring valve 9 of valve 7 such that, in use, when the end of the limb 20 is pushed through the ring valve 9 a liquid-tight connection is made between the tube 12 and the ring valve 9. On travelling further into the valve 7 the end of the limb 20 will meet the slit valve 11 and force it open. Since the limb 20 and the ring valve 9 have formed a liquid-tight connection any liquid that flows past the slit valve 11 will be prevented from leaking out of the refill 1. Once the limb 20 is inside the refill as shown in FIG. 7, liquid 4 is able to flow through the perforation(s) into the hollow interior of the limb 20 and flow in a downward direction powered by gravity and into the device. The tube 12 permits air to flow into the reservoir in the head-space formed by the dropping liquid level to prevent any retarding of the transport of liquid out of the refill 1 due to build up of negative pressure as liquid 4 leaves the reservoir 3.

Although not shown, the end of the housing 1 containing the valve 7 or valves 7,8 may be substantially square in shape and guide means (not shown) in a chassis (not shown) of each emanation device) could cooperate with grooves in the refill housing (not shown) to ensure that the refill could only be engaged with the chassis if the limb 11 is correctly aligned to open the valve 7 or valves 7,8. Although not illustrated, the end of the refill housing containing the valve 7 or valves 7,8 could be shaped such that the loading thereof into the chassis was only possible via a single orientation of that end of the refill.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A liquid dispensing system comprising a universal refill of liquid and an emanation device; wherein the refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick; and
wherein the emanation device is selected from: an emanation device configured to spray the liquid; an emanation device configured to evaporate the liquid by electrically powered means; an emanation device configured to evaporate the liquid by passive, non-electrically powered means; an emanation device configured to emanate the liquid by two or more of spraying the liquid, evaporating the liquid by electrically powered means and/or evaporating the liquid by passive, non-electrically powered means;
characterised in that each emanation device is provided with a chassis configured to receive and releasably retain the universal refill and the universal refill is operationally compatible with each emanation device.

2. A liquid dispensing system comprising a universal refill of liquid and an emanation device; wherein the refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick; and
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill and the universal refill is operationally compatible with each emanation device.

3. A liquid dispensing device for use with a universal refill of liquid and an emanation device; wherein the refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick;
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill.

4. A liquid dispensing system according to claim 1, wherein the universal refill is provided with securing means which are configured, in use, to cooperate with the chassis and/or the emanation device to permit secure engagement therewith.

5. A liquid dispensing system according to claim 1, wherein the universal refill housing has a single aperture.

6. A liquid dispensing system according to claim 1, wherein the universal refill provided with one or more vent holes in the housing, said vent holes being provided in the form of a one-way valve that is in communication with the reservoir but does not facilitate a liquid pathway therefrom

7. A liquid dispensing system according to claim 1, wherein the universal refill further comprises one or more diptubes therein.

8. A liquid dispensing system according to claim 1, wherein the wick(s) is provided on or connected to the outer surface of the upper wall of the housing adjacent the aperture(s).

9. A liquid dispensing system according to claim 1, wherein the wick(s) is located on or connected to the outer surface of the housing such that when the refill is located in a position to extract and emanate liquid contained within the reservoir, then said at least one wick is located substantially level with or above the liquid level in the reservoir.

10. A liquid dispensing system according to claim 1, wherein more than one wick is provided on or in contact with numerous outer surfaces of the housing.

11. A liquid dispensing system according to claim 1, wherein, the wick is provided on a collar which is connected to the outer surface of the housing and the collar is sized to extend away from the outer surface of the housing.

12. A liquid dispensing system according to claim 1, wherein the chassis is formed as a separate component to the emanation device and is mounted on or in the device to receive and releasably retain the universal refill.

13. A liquid dispensing system according to claim 1, wherein the chassis is formed in an exterior surface of the emanation device.

14. A liquid dispensing system according to claim 1, wherein the chassis is formed within the interior of the device and a suitably shaped aperture is provided to permit access to the interior of the housing by the universal refill.

15. A liquid dispensing system according to claim 1, wherein the chassis is formed within the interior of the device and the device is provided with a door which may be opened to permit access to the interior of the housing by the universal refill.

16. A liquid dispensing system according to claim 1, whereby when the emanation device is to pump the liquid from the reservoir(s) of the universal refill the emanation device is provided with one or more liquid conduits that are connected at one end to a pumping means housed within the device and open at the other end to contact the liquid, and wherein the liquid conduit(s) are sized to extend through the, or each, valve system in the universal refill and into the reservoir(s) therewithin when the refill is engaged with the chassis such that, in use, the pump is operable via conventional pumping mechanisms to create negative pressure within the liquid conduit(s) in order to draw the liquid into the conduit(s) and transport same toward the pump.

17. A liquid dispensing system according to claim 1, whereby when the emanation device is to blow the liquid from the reservoir(s) of the universal refill the emanation device is provided with one or more blown air conduits that are connected at one end to a blowing means housed within the device and open at the other end to permit blown air to exit therefrom and with one or more liquid conduits that are open at one end to contact the liquid and connected to the device at the other end, and wherein the blown air conduit(s) are sized to enter through one valve system of the universal refill and the liquid conduit(s) are sized to extend through one or more of alternate valve systems in the refill and wherein both types of conduits extend into the reservoir(s) within the refill when said refill is engaged with the chassis such that, in use, the blowing means is operable to blow air along the blown air conduit and into the reservoir(s) in order to pressurise the head space and, when required by the device, force liquid into the liquid conduit(s) and toward the device.

18. A liquid dispensing system according to claim 1, whereby when the universal refill is provided with a single aperture and single valve system, the blown air conduit and liquid conduit are integrated such that the blown air conduit extends along a central core of the liquid conduit with the blown air conduit being provided with the greater length such that it extends further into the refill and such that the conduits enter the single aperture and valve system, and wherein the blown air conduit, in use, blows air into the headspace of the refill to force liquid into the liquid conduit and into the device for emanation therefrom.

19. A liquid dispensing system according to claim 1, whereby when the emanation device is to wick the liquid from the reservoir(s) of the universal refill the emanation device is provided with one or more wicks that are connected at one end to the emanation device and configured at the other end to contact the liquid and uptake same, and wherein the wick(s) are sized to extend through the valve system(s) in the universal refill and into the reservoir(s) therewithin when the refill is engaged with the chassis such that, in use, the wick(s) are operable to passively uptake liquid from the reservoir(s) and transport same towards the other end of the wick(s).

20. A liquid dispensing system according to claim 1, whereby when the emanation device is to transport the liquid from the reservoir(s) of the universal refill to the emanation device via capillary action the device is provided with one or more capillary tubes that contact the liquid and uptake same at one end and terminate at their other end adjacent to or in contact with the wick on the exterior surface of the universal refill housing, and wherein the capillary tube(s) are sized to extend through the valve system(s) in the universal refill and into the reservoir(s) therewithin when the refill is engaged with the chassis such that, in use, the capillary tube (s) are operable to passively uptake liquid from the reservoir(s) and transport same towards the other end of the tube(s) and on to the wick on the exterior of the refill housing, and wherein a heater in the device is located adjacent the wick(s) to, in use, direct heat toward to the wick(s) to accelerate the emanation of the liquid therefrom.

21. A liquid dispensing system according to claim 1, whereby when the emanation device is to extract the liquid from the reservoir(s) of the universal refill toward the emanation device by a gravity feed mechanism the emanation device is provided with one or more liquid conduits that are connected at one end to the device and open at the other end to contact the liquid, and wherein the liquid conduit(s) are sized to extend through the valve system(s) in the universal refill and into the reservoir(s) therewithin when the refill is engaged with the chassis, and wherein the chassis is orientated such when the universal refill is engaged therewith the valve system(s) of the refill are located closer to a supporting surface on which the device is held such that once the valve(s) are opened by the liquid conduit(s) the force of gravity will cause liquid to travel from the refill toward the device.

22. A liquid dispensing system according to any claim 1, wherein the emanation device and/or chassis is provided with securing means which are configured to engage with the universal refill to permit a secure engagement of the universal refill to the chassis.

23. A liquid dispensing system according to claim 1, wherein the universal refill and emanation devices are provided with a device-to-refill recognition system whereby the device is able to recognise the level of liquid remaining in the refill and/or the type of liquid in the refill and/or whether the refill is from a known source.

24. A universal refill of liquid containing one or more active materials wherein the refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick; and
wherein the refill is configured to allow the liquid to be extracted by at least two different methods of liquid extraction.

25. A universal refill containing a liquid for use with at least two different types of emanation device; wherein the universal refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick; and
wherein each type of device emanates the liquid from the universal refill via a different mechanism to each other type of emanation device and wherein said at least two different types of emanation device each comprise a chassis configured to receive and releasably retain the universal refill.
